(19) United States
(12) Patent Application Publication
(10) Pub. No.: US 2024/0271085 A1
(43) Pub. Date: Aug. 15, 2024

(54) ACTIVATED DISSOLVABLE SUPPORTS FOR AFFINITY BINDING AND CELL CULTURE

(71) Applicant: CORNING INCORPORATED, Corning, NY (US)

(72) Inventors: David Henry, Fontaine le Port (FR); Corinne Walerack, Veneux les sablons (FR)

(21) Appl. No.: 18/028,961

(22) PCT Filed: Sep. 14, 2021
(86) PCT No.: PCT/US2021/050221

§ 371 Date: Mar. 28, 2023

Related U.S. Application Data
(60) Provisional application No. 63/084,153, filed on Sep. 28, 2020.

(51) Int. Cl. C12N 5/00

(52) U.S. Cl. CPC C12N 5/0068; C12N 2533/40; C12N 2537/10; C12N 2539/10

(57) ABSTRACT

Activated dissolvable supports are provided comprising an ionotropically crosslinked compound comprising a polymer material having at least one repeating unit comprising an ionically crosslinked carboxylic acid group, and an activated hydroxyl group, wherein the hydroxyl group is activated by N,N′-disuccinimidyl carbonate (DSC) or N-hydroxy succinimidyl chloroformate in a solvent to form succinimidyl carbonate groups for ligand binding. Methods of forming activated dissolvable supports, culturing cells on activated dissolvable supports, and harvesting cells from dissolvable supports are provided.

### CROSS-REFERENCE TO RELATED APPLICATIONS

This application claims the benefit of priority under 35 U.S.C. § 119 of U.S. Provisional Application Ser. No. 63/084,153 filed on Sep. 28, 2020, the content of which is relied upon and incorporated herein by reference in its entirety.

### TECHNICAL FIELD

This disclosure generally relates to activated dissolvable supports. In particular, the present disclosure relates to activated dissolvable supports for use in cell culture and cell capture.

### BACKGROUND

Cell therapies are gaining popularity as treatment methods for many diseases. In conventional cell therapy production, methods are labor-intensive and are formatted for production in small batches. However, many emerging cell therapies require the isolation of low-frequency cells of interest from a heterogeneous cell mixture, followed by large-scale expansion of the captured cells. Conventional small-batch production techniques do not rise to meet the increasing need for cell therapy production methods that allow for isolation of target cells and efficient culture of the cells.

In conventional cell isolation techniques, ligand-receptor interactions are used to isolate cells, and the ligands must be immobilized on a solid support. However, conventional methods of immobilizing ligands on a solid support rely on the activation of carboxylic acid groups, which impacts the mechanical integrity of the support and may lead to degradation of the support during further processing.

### SUMMARY

Embodiments of the disclosure provide activated dissolvable supports. The supports allow for cell culture, cell capture, or both while maintaining mechanical integrity in cell culture medium. The dissolvable supports do not require a polymer adhesion coating, as the ligands that permit cell capture or cell culture are covalently immobilized and thus durably attached to the dissolvable support. Due to the nature of the ionic crosslinking within the dissolvable support, the support maintains mechanical integrity in the presence of culture media.

Because the supports are dissolvable and thus can disappear on-demand, supports according to embodiments of the disclosure allow for downstream processing to be simplified. Thus, the dissolvable support provides for enhanced recovery of the captured cells or the cultured cells because the support itself is dissolvable on demand, such as by digestion from addition of an enzyme. In addition, the dissolvable supports can be in any suitable format (e.g., beads, fibers, foam monolith, among others) and are not limited to cell culture on a planar surface, such as in two-dimensional (2D) monolayer cell culture.

In an aspect, an activated dissolvable support comprises an ionotropically crosslinked compound comprising a polymer material having at least one repeating unit comprising: an ionically crosslinked carboxylic acid group, and an activated hydroxyl group, wherein the hydroxyl group is activated by N,N′-disuccinimidyl carbonate (DSC) or N-hydroxysuccinimidyl chloroformate in a solvent to form succinimidyl carbonate groups for ligand binding.

In some embodiments, the carboxylic acid group is ionically crosslinked with a multivalent cation. In some embodiments, the solvent is an aprotic solvent. In some embodiments, the aprotic solvent is an anhydrous solvent.

In some embodiments, the at least one repeating unit comprises:

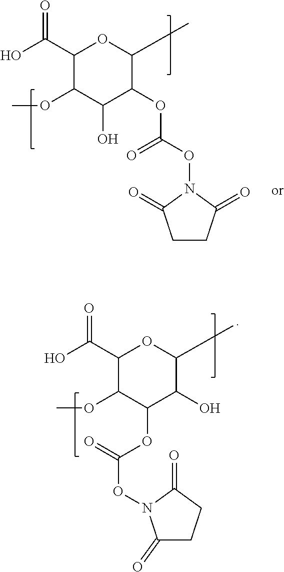

In some embodiments, the polymer material comprises a polygalacturonic acid (PGA) compound. In some embodiments, the PGA compound comprises at least one of pectic acid, partially esterified pectic acid, partially amidated pectic acid, or salts thereof. In some embodiments, the partially esterified pectic acid comprises a degree of esterification from 1 mol % to 40 mol %. In some embodiments, the partially amidated pectic acid comprises a degree of amidation from 1 mol % to 40 mol %.

In some embodiments, the dissolvable support comprises a structure comprising beads, fibers, fabric, foam, or a coating. In some embodiments, the dissolvable support comprises porous beads. In some embodiments, the dissolvable support comprises a macroporous foam. In some embodiments, the dissolvable support comprises a coating for cell culture surfaces of cell culture vessels.

In some embodiments, the dissolvable support is dissolved by digestion from an enzyme, chelating agent, or a combination thereof. In some embodiments, the enzyme comprises a non-proteolytic enzyme. In some embodiments, the non-proteolytic enzyme is selected from the group consisting of pectinolytic enzymes and pectinases. In some embodiments, digestion of the dissolvable support is complete in less than about 1 hour. In some embodiments, digestion of the dissolvable support is complete in less than about 15 minutes. In some embodiments, the ligand comprises a protein, peptide, peptoid, sugar, or drug.

In an aspect, a method of forming an activated dissolvable support comprises forming an ionotropically crosslinked compound comprising: forming a dissolvable support by adding a polymer solution comprising a polygalacturonic acid (PGA) compound to a solution comprising at least one multivalent cation, the PGA compound selected from at least one of: pectic acid, partially esterified pectic acid, partially amidated pectic acid and salts thereof; and activating the hydroxyl groups to form succinimidyl carbonate groups in the PGA compound of the dissolvable support by adding an activation solution.

In some embodiments, the method further comprises rinsing the dissolvable support before activation to remove unbound hydroxyl-containing compounds. In some embodiments, the method further comprises coupling ligands to the activated hydroxyl groups to create a ligand-dissolvable support conjugate. In some embodiments, the ligand comprises proteins, peptides, peptoids, sugars, and drugs. In some embodiments, the ligand-dissolvable support conjugate comprises at least one unit comprising:

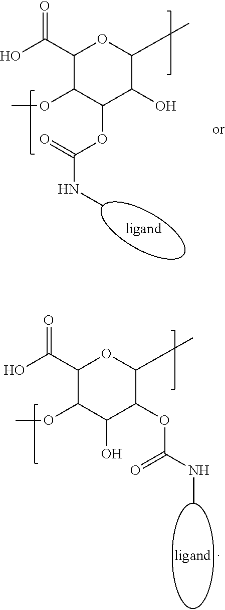

In some embodiments, the method further comprises rinsing the activated dissolvable support after activation with a solution that contains at least one multivalent cation.

In some embodiments, the ionotropically crosslinked compound can be formed into a structure comprising beads, fibers, fabric, or a foam. In some embodiments, the ionotropically crosslinked compound can be applied to a cell culture surface as a coating.

In some embodiments, the activation solution comprises an activation agent and a solvent. In some embodiments, the activation agent comprises N,N′-disuccinimidyl carbonate (DSC) or N-hydroxysuccinimidyl chloroformate. In some embodiments, the solvent comprises an aprotic solvent.

In some embodiments, the polygalacturonic acid compound comprises at least one of pectic acid, partially esterified or amidated pectic acid having a degree of esterification or amidation from 1 to 40 mol %, or salts thereof. In some embodiments, the polygalacturonic acid compound contains less than 20 mol % methoxyl groups.

In an aspect, a method for culturing cells on a dissolvable support comprises seeding cells on a dissolvable support; and contacting the dissolvable support with cell culture medium.

In some embodiments, seeding cells on a dissolvable support comprises adhering cells to the surface of the dissolvable support. In some embodiments, cells aggregate in pores of the dissolvable support to form spheroids.

In some embodiments, contacting the dissolvable support with cell culture medium comprises submerging the dissolvable support in cell culture medium. In some embodiments, contacting the dissolvable support with cell culture medium comprises continuously passing cell culture medium over the dissolvable support. In some embodiments, continuously passing cell culture medium over the dissolvable support comprises removing at least some of the cell culture medium from contact with the dissolvable support and contacting the dissolvable support with fresh cell culture medium such that the volume of cell culture medium in contact with the dissolvable support remains substantially constant.

In an aspect, a method of harvesting cells from a dissolvable support comprises digesting the dissolvable support by exposing the dissolvable foam scaffold to an enzyme, chelating agent, or a combination thereof; and harvesting cells exposed when the dissolvable support is digested.

In some embodiments, the dissolvable support comprises an ionotropically crosslinked polygalacturonic acid compound selected from at least one of: pectic acid; partially esterified pectic acid, partially amidated pectic acid and salts thereof, and wherein the enzyme comprises a non-proteolytic enzyme. In some embodiments, the non-proteolytic enzyme is selected from the group consisting of pectinolytic enzymes and pectinases. In some embodiments, digesting the dissolvable support comprises exposing the dissolvable support to between about 1 U and about 200 U of the enzyme. In some embodiments, digesting the dissolvable support comprises exposing the dissolvable support to between about 1 mM and about 200 mM of the chelating agent.

### BRIEF DESCRIPTION OF THE DRAWINGS

FIG. 1 is a fluorescent image of cells seeded in a support according to an embodiment of the disclosure.

FIG. 2 is a fluorescent image of cells seeded in a support according to an embodiment of the disclosure.

FIG. 3 is a fluorescent image of cells seeded in a negative control described in Example 5.

FIG. 4 is a fluorescent image of cells captured in a support according to an embodiment of the disclosure.

FIG. 5 is a fluorescent image of cells captured in a negative control described in Example 6.

### DETAILED DESCRIPTION

Embodiments of the disclosure provide activated dissolvable supports. The supports allow for cell culture, cell capture, or both while maintaining mechanical integrity in cell culture medium. Furthermore, the ligands that permit cell capture or cell culture are covalently immobilized and thus durably attached to the dissolvable support. Because the supports are dissolvable and thus can disappear on-demand, supports according to embodiments of the disclosure allow for downstream processing to be simplified.

In an aspect of the invention, an activated dissolvable support is provided. The dissolvable support comprises an ionotropically-crosslinked material that is dissolvable. In some embodiments, the dissolvable material comprises pectinic or pectic acid derivatives or alginic acid derivatives, such as described in WO2014209865A1 and WO2019104069, the contents of which are incorporated herein in their entirety.

Conventional activation methods rely on the activation of carboxylic acid groups. In contrast to the conventional activation methods, the hydroxyl activation method in embodiments of the disclosure leave the carboxyl (COOH) groups, which are involved in ionotropic gelation, unaffected. Therefore, embodiments of the disclosure preserve the mechanical integrity of the support that can be further processed without any risk of degradation since the density of ionic crosslinking is not altered. In embodiments of the disclosure, dissolvable supports may be activated and stored for months for further ligand coupling with minimum risk of immobilization capacity-loss due to the high stability of the carbonate linkage.

Traditional coupling of ligands through their amine functional groups onto dissolvable pectinic acid derivatives, pectic acid derivatives, or alginic acid derivatives is performed by means of two main methods. The first conventional method relies on activation of carboxylic acid groups that are converted into activated ester, e.g. NHS ester, and formation of amide bond between the support and the ligand to be immobilized. Such carbodiimide-based chemistry is the chemistry of choice for polymers containing carboxylic acid groups, carboxymethyl cellulose, alginic acid, hyaluronic acid, and polyacrylic acid, among others, and is used for coupling amine-containing compounds to alginate or pectin derivatives, which are both gelling polymers bearing numerous carboxyl groups. The second conventional method involves creating aldehyde groups by controlled oxidation of cis-diols on the polysaccharide backbone using periodic acid or periodate. Such methods are used for the activation of polysaccharides bearing carboxylic acid groups, such as alginic acid or other carboxylic acid-containing biopolymers.

However, conventional techniques result in a significant loss of mechanical properties in the polymeric support. In the case of the carbodiimide-mediated coupling, the consumption of carboxylic acid groups involved in the ionic crosslinking to form the activated ester leads to a drastic reduction of the crosslinking density. The reduction of crosslinking density consequently leads to degraded mechanical properties of the support, and the support is no longer able to resist agitation, flow of cell culture medium, or flow required for affinity capture within a column format. In the case of the aldehyde-activated supports, the introduction of aldehyde groups by controlled oxidation using periodate or periodic acid leads to a significant reduction of the molecular weight of the polymer and some oxidative decarboxylation, which may result in a drastic reduction of the mechanical resistance of the support. Thus, conventional techniques do not allow for activation of the dissolvable support without degrading its crosslinking density and while also providing an acceptable dimensional stability.

The present disclosure relates to an activated dissolvable ionically-crosslinked support for affinity capture of cells, cell culture, or a combination thereof. The activated support can be used for immobilization of ligands and subsequent capture of molecules or cells. The support is particularly suited to immobilize ligands such as peptides and proteins and to perform cell sorting or cell culture. The activated support is dissolvable on-demand. For example, the support can be eliminated or dissolved on-demand by simple addition of enzymes and/or chelating agents. The dissolvability is advantageous for recovery of the molecules or cells that have been captured by affinity. A full dissolution also facilitates the purification of the captured target.

Methods according to embodiments of the disclosure rely on the activation of the hydroxyl groups from the ionically-crosslinked dissolvable support. The activation is by means of N,N′-disuccinimidyl carbonate (DSC) or N-hydroxysuccinimidyl chloroformate in a solvent to form succinimidyl carbonate groups that are highly reactive toward amine nucleophiles and able to form durable carbamate linkages. In some embodiments, the solvent may be an anhydrous solvent.

In embodiments, hydroxyl groups are activated by means of N,N′-disuccinimidyl carbonate (DSC) or N-hydroxylsuccinimidyl chloroformate in an anhydrous solvent to form succinimidyl carbonate groups. Such activation of the hydroxyl groups prevents loss of the mechanical integrity of the dissolvable supports while allowing for efficient immobilization of ligands.

In embodiments, the dissolvable material is preferably made of alginate or pectin derivatives, such as pectate and pectinate, that are ionically-crosslinked (ionotropically crosslinked). Such physical crosslinking relies on interaction of carboxylic acid groups with multivalent cations such as calcium. Such a physical crosslinking is reversible, and the crosslinking of the material can be disrupted by contacting it with chelating agents. In some embodiments, the material can be fully digested by adding enzymes. Among enzymes, pectinase and alginate lyase can be used to digest pectate or pectinate and alginate materials, respectively.

By using coupling on hydroxyl groups instead of carboxyl groups, dissolvable supports according to embodiments of the disclosure maintain a high ionic crosslinking density and eliminate the risks of mechanical integrity or geometry loss of the support.

In one aspect, the polymeric material forming the activated-dissolvable support comprises at least one unit comprising:

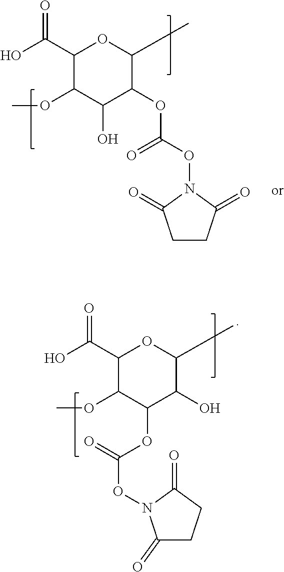

In another aspect, the ligand-dissolvable support conjugate comprises at least one unit comprising:

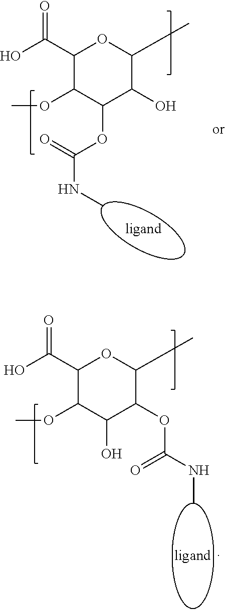

The ligand may be any synthetic or natural molecules bearing at least one amino group and being able to interact with the target cell. Preferred ligands are proteins, peptides, peptoids, specific sugars, and drugs, among others.

The geometry of the activated ionically-crosslinked support may have any suitable geometry. In some embodiments, the activated ionically-crosslinked support comprises beads, fibers, fabrics, or foam monoliths. In some embodiments, the supports comprise macroporous foams, or porous beads. In some embodiments, the dissolvable supports are configured to be disposed within a cartridge or vessel for cell culture and may be configured to fill the cartridge volume accordingly. As an example, when the dissolvable support is in the form of a piece of macroporous foam, the activation of the foam support preserves the geometry of the foam which is required to properly fit the volume of the cartridge in which the pieces of foam are placed.

In some embodiments, the dissolvable support is macroporous, which allows for easy flow of liquid throughout the material with low backpressure and makes the separation processes easier. Macroporous materials can be prepared by any suitable method, such as effervescence, gas foaming, and aeration by whipping, among others.

Methods according to embodiments of the disclosure are useful to couple ligands to highly porous foams made of polysaccharides bearing COOH groups, such as pectin derivatives, despite the acidic nature of the foams. The method is advantageous for functionalizing highly porous foams that provide easy flow through, but may lead to dimensional stability issues due to the low amount of solid matter present in the material (typically less than 2 wt. %) and their hydrogel nature and thus an inherent low modulus due to its ability to absorb large amount of water. Due to the weak mechanical properties, preparation of large size separation columns remains challenging. Consequently, a coupling chemistry that maintains a high ionic crosslinking as the one described in the present disclosure helps in keeping acceptable mechanical resistance and dimensional stability.

Any suitable polymer or biopolymer may be used for the dissolvable support and used in methods of forming a dissolvable support according to embodiments of the disclosure. In some embodiments, the biopolymers comprise polysaccharides, which are hydrophilic, non-cytotoxic, and stable in culture medium. Dissolvable supports as described herein may comprise at least one ionotropically crosslinked polysaccharide. Generally, polysaccharides possess attributes beneficial to cell culture applications. Polysaccharides are hydrophilic, non-cytotoxic and stable in culture medium. Examples include pectic acid, also known as polygalacturonic acid (PGA), or salts thereof, partly esterified pectic acid or salts thereof, or partly amidated pectic acid or salts thereof. Pectic acid can be formed via hydrolysis of certain pectin esters. Pectins are cell wall polysaccharides and in nature have a structural role in plants. Major sources of pectin include citrus peel (e.g., peels from lemons and limes) and apple peel. Pectins are predominantly linear polymers based on a 1,4-linked alpha-D-galacturonate backbone, interrupted randomly by 1,2-linked L-rhamnose. The average molecular weight ranges from about 50,000 to about 200,000 Daltons.

Alginate is an example polysaccharide polymer material for forming a dissolvable support or cell culture scaffold. Alginate is a binary copolymer of 1-4 linked β-D mannuronic acid (M) and α-L guluronic (G). The monomers are arranged in pattern blocks along the chain, and divalent cations (Ca2+, Ba2+, Sr2+) bind preferentially to G blocks in the alginate and form bonds between adjacent alginate chains. Consequently, the stability of the crosslinked gel depends on the amount of G blocks.

In some embodiments, polygalacturonic acid (PGA) polymers are used to form a dissolvable support or cell culture scaffold. In the case of polygalacturonic acid, or pectic acid, each monomer unit may potentially be involved in ionic crosslinking, which leads to highly crosslinked gels. The high crosslinking ability makes PGA attractive in term of mechanical properties and high stability, especially when exposed to a high ionic strength medium like a medium usually encountered in cell culture. In addition, a higher solid content can be achieved with PGA since the viscosity of PGA solutions is usually lower than those made from alginates.

In embodiments, the dissolvable support comprises a polygalacturonic acid compound. In embodiments, the polygalacturonic acid compound comprises at least one of pectic acid, partially esterified or amidated pectic acid having a degree of esterification or amidation from 1 to 40 mol %, or salts thereof.

Polygalacturonic acid results from the controlled hydrolysis of pectins which are cell wall polysaccharides which have a structural role in plants. They are predominantly linear polymers based on a 1,4-linked alpha-D-galacturonate backbone, interrupted randomly by 1,2-linked L-rhamnose. The average molecular weight is from about 50,000 to about 200,000 Daltons. Two major sources of pectins are, for example, from citrus peel (mostly lemon and lime) or apple peels and can be obtained by extraction thereof.

The polygalacturonic acid chain of pectins can be partly esterified, and methyl groups and the free acid groups may be partly or fully neutralized with monovalent ions such as sodium, potassium, or ammonium ions. Pectinic acids are polygalacturonic acids partly esterified with methanol, and salts thereof are called pectinates. The degree of methylation (DM) for commercial high methoxyl (HM) pectins typically can be, for example, from about 60 to about 75 mol % and those for low methoxyl (LM) pectins can be from about 1 to about 40 mol %, about 10 to about 40 mol %, and about 20 to about 40 mol %, including intermediate values and ranges.

In embodiments, the dissolvable support is preferably prepared from LM pectins. In embodiments, the polygalacturonic acid contains less than 20 mol % methoxyl groups. In embodiments, the polygalacturonic acid has no or only negligible methyl ester content as pectic acids. For simplicity, both pectinic acids having no or only negligible methyl ester and low methoxyl (LM) pectins are referred to as PGA in the disclosure. For the same reasons, when the pectic or pectinic acid is amidated, the degree of amidation must be low enough to enable crosslinking by ionotropic gelation.

The polygalacturonic acid chain of pectin may be partly amidated. Polygalacturonic acids partly amidated pectin may be produced, for example, by treatment with ammonia. Amidated pectin contains carboxyl groups (˜COOH), methyl ester groups (˜COOCH₃), and amidated groups (—CONH₂). The degree of amidation may vary and may be, for example, from about 10% to about 40% amidated.

According to embodiments of the present disclosure, dissolvable supports as described herein may include a mixture of pectic acid and partly esterified pectic acid. Blends with compatible polymers may also be used. For example, pectic acid and/or partly esterified pectic acid may be mixed with other polysaccharides such as dextran, substituted cellulose derivatives, alginic acid, starches, glycogen, arabinoxylans, agarose, etc. Glycosaminoglycans like hyaluronic acid and chondroitin sulfate, or various proteins such as elastin, fibrin, silk fibroin, collagen and their derivatives can be also used. Water soluble synthetic polymers can be also blended with pectic acid and/or partly esterified pectic acid. Exemplary water-soluble synthetic polymers include, but are not limited to, polyalkylene glycol, poly(hydroxyalkyl(meth)acrylates), poly(meth)acrylamide and derivatives, poly(N-vinyl-2-pyrrolidone), and polyvinyl alcohol.

In some embodiments, the polygalacturonic acid compound comprises at least one of pectic acid, partially esterified or amidated pectic acid having a degree of esterification or amidation from 1 to 40 mol %, or salts thereof. In some embodiments, the polygalacturonic acid compound contains less than 20 mol % methoxyl groups.

In an aspect, a method of forming an activated dissolvable support comprises forming an ionotropically crosslinked compound comprising: forming a dissolvable support by adding a polymer solution comprising a polygalacturonic acid (PGA) compound to a solution comprising at least one multivalent cation, the PGA compound selected from at least one of: pectic acid, partially esterified pectic acid, partially amidated pectic acid and salts thereof; and activating the hydroxyl groups in the PGA compound of the dissolvable support by adding an activation solution.

In some embodiments, the activated dissolvable support can be formed into a structure comprising beads, fibers, fabric, or a foam. In some embodiments, the activated dissolvable support can be applied to a cell culture surface as a coating.

The dissolvable supports according to embodiments of the disclosure may be crosslinked to prevent their dissolution into the cell culture medium. In some embodiments, methods comprise crosslinking by ionotropic gelation of pectic acid derivatives such as PGA. Crosslinking is preferably performed by ionotropic gelation. Ionotropic gelation is based on the ability of polyelectrolytes to crosslink in the presence of multivalent counter ions to form crosslinked hydrogels. Crosslinking can be performed by external ionotropic gelation. Various divalent cations can be used for crosslinking, including the non-limiting examples of calcium, strontium, or barium.

In some embodiments, the activation solution comprises an activation agent and a solvent. Any suitable activation agents may be used in methods according to embodiments of the disclosure. In some embodiments, the activation agent is N,N′-disuccinimidyl carbonate (DSC). In some embodiments, the activation agent is N-hydroxysuccinimidyl chloroformate. Any suitable solvent may be used in methods according to embodiments of the disclosure. In some embodiments, the solvent comprises an aprotic solvent. Because aprotic solvents do not have O—H or N—H bonds, side reactions may be avoided. In some embodiments, the aprotic solvent comprises an anhydrous solvent. Non-limiting examples of aprotic solvents include anhydrous acetone, anhydrous DMSO, anhydrous NMP, and anhydrous DMAc, among others. In some embodiments, the solvent is anhydrous DMSO. In some embodiments, the aprotic solvent is water miscible. The water miscibility of the solvent prevents an excessive shrinkage of the support upon activation, such as when activation is performed on a macroporous support that may be used within a column format.

In some embodiments, the method further comprises rinsing the dissolvable support before activation to remove unbound hydroxyl-containing compounds. The support may be carefully washed before activation in order to remove all unbound hydroxyl-containing compounds that could react with the succinimidyl carbonate reagent and therefore decrease the activation of the PGA. The hydroxyl-containing compounds may be present in PGA porous foam as foaming additives, such as the non-limiting examples of low molecular weight sugars, plasticizers such as glycerol, and surfactants. In embodiments, the rinsing step is performed with water followed by rinsing with a dry solvent such as DMSO. The extent of water remaining in the material can affect the level of activation. Extensive washing with the anhydrous solvent leads to a higher activation degree.

In some embodiments, methods according to embodiments of the disclosure further comprise a rinsing step after activation. In some embodiments, the method further comprises rinsing the activated dissolvable support after activation with a solution that contains at least one multivalent cation. In order to avoid a decrease of ionic crosslinking, the rinsing step after activation and coupling of the ligand are preferably performed using a solution that contains at least one multivalent ion, e.g. calcium. As a non-limiting example, a CaCl2 solution is a solution that contains at least one multivalent ion.

In some embodiments, the method further comprises coupling ligands to the activated hydroxyl groups to create a ligand-dissolvable support conjugate. In some embodiments, the ligand comprises proteins, peptides, peptoids, sugars, and drugs. In some embodiments, the ligand-dissolvable support conjugate comprises at least one unit comprising:

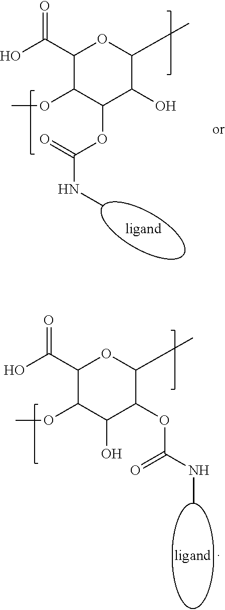

### Cell Culture, Cell Capture, and Cell Harvesting

Recent research suggests that in contrast to two-dimensional (2D) or monolayer culture, the environment experienced by cells in-vivo is more accurately represented by 3D cell culture, and it has been demonstrated that cell responses in 3D cultures are more similar to in-vivo behavior than the cell responses in 2D cultures. The additional dimensionality of 3D cultures is believed to lead to the differences in cellular responses because it influences the spatial organization of the cell surface receptors engaged in interactions with surrounding cells and induces physical constraints to cells, thereby affecting the signal transduction from the outside to the inside of cells, ultimately influencing gene expression and cellular behavior.

Cell culture technologies have been developed in order to simulate the natural 3D environment of cells. For instance, some bioreactors include a carrier in the form of a stationary packing material forming a fixed or packed bed for promoting cell adhesion and growth. Another example of 3D cell culture technology is a porous 3D matrix or scaffold which promotes the growth and proliferation of the cultured cells within pores and other interior spaces of the matrix. However, such technologies often use a protease treatment to harvest the cells, thereby subjecting the cells to harsh conditions which may damage cell structure and function. Additionally, using protease treatment often causes only a limited amount of cell detachment. For the fixed bed material, the densely packed nature of the fixed bed material makes it more difficult to circulate the protease agent throughout the bed and increase the yield of cells harvested. Similarly, it can be difficult to circulate the protease agent through interior spaces of the 3D matrix, which in turn makes it difficult to dislodge cells during the harvest process. This difficulty is compounded by the presence of extracellular macromolecules secreted by the cultured cells that serve to attach the cells to the surface of the fixed bed material or to the surface of the matrix. Therefore, mechanical force has been used in conventional cell culture technologies, either alternatively, or in combination with the protease treatment to harvest cells. Such methods and systems for harvesting cells apply mechanical force to release cultured cells from the fixed bed material or the 3D matrix. For example, the fixed bed material, 3D matrix, or a larger system including the fixed bed material or the 3D matrix, may be shaken or vibrated to release the cultured cells. However, use of mechanical force may also cause physical damage to the cultured cells, which reduces cell culture yields.

According to embodiments of the present disclosure, methods for culturing cells or capturing cells on dissolvable supports as described herein are also disclosed. In some embodiments, methods comprise cell capture or cell culture of cell aggregates, or spheroids, in dissolvable supports. In some embodiments, methods comprise cell culture in dissolvable supports within bioreactor systems. Any type of cell may be cultured on the dissolvable supports including, but not limited to, immortalized cells, primary culture cells, cancer cells, stem cells (e.g., embryonic or induced pluripotent), etc. The cells may be mammalian cells, avian cells, piscine cells, etc. The cells may be of any tissue type including, but not limited to, kidney, fibroblast, breast, skin, brain, ovary, lung, bone, nerve, muscle, cardiac, colorectal, pancreas, immune (e.g., B cell), blood, etc. The cells may be in any cultured form including disperse (e.g., freshly seeded), confluent, 2-dimensional, 3-dimensional, spheroid, etc. Culturing cells on a dissolvable support may include seeding cells on the dissolvable supports. Seeding cells on a dissolvable support may include contacting the support with a solution containing the cells. The activated supports according to embodiments of the disclosure are customizable and may be functionalized with different components or compounds that promote cell adhesion (e.g., proteins, peptides). Once the seeded cells are introduced to the functionalized support, the cells adhere to the surface of the functionalized support.

Culturing cells on dissolvable supports may further include contacting the supports with cell culture medium. Generally, contacting the supports with cell culture medium includes placing cells to be cultured on the supports in an environment with medium in which the cells are to be cultured. Contacting the supports with cell culture medium may include pipetting cell culture medium onto the supports, or submerging the supports in cell culture medium, or passing cell culture media over the supports in a continuous manner. Generally, as used herein, the term “continuous” refers to culturing cells with a consistent flow of cell culture medium into and out of the cell culture environment. Such passing cell culture media over the supports in a continuous manner may include submerging the supports in cell culture medium for a predetermined period of time, then removing at least some of the cell culture medium after the predetermined period of time and adding fresh cell culture medium such that the volume of cell culture medium in contact with the dissolvable supports remains substantially constant. Cell culture medium may be removed and replaced according to any predetermined schedule. For example, at least some of the cell culture medium may be removed and replaced every hour, or every 12 hours, or every 24 hours, or every 2 days, or every 3 days, or every 4 days, or every 5 days.

Any cell culture medium capable of supporting the growth of cells may be used. Cell culture medium may be for example, but is not limited to, sugars, salts, amino acids, serum (e.g., fetal bovine serum), antibiotics, growth factors, differentiation factors, colorant, or other desired factors. Exemplary cell culture medium includes Dulbecco's Modified Eagle Medium (DMEM), Ham's F12 Nutrient Mixture, Minimum Essential Media (MEM), RPMI Medium, Iscove's Modified Dulbecco's Medium (IMDM), MesenCult™-XF medium (commercially available from STEMCELL Technologies Inc.), and the like.

According to embodiments of the present disclosure, methods for harvesting cells from dissolvable supports as described herein are also disclosed.

Dissolvable supports as disclosed herein are described as being dissolvable and insoluble. As used herein, the term “insoluble” is used to refer to a material or combination of materials that is not soluble, and that remains crosslinked, under conventional cell culture conditions which include, for example, cell culture media. Also as used herein, the term “dissolvable” is used to refer to a material or combination of materials that is digested when exposed to an appropriate concentration of an enzyme and/or chelating agent that digest or break down the material or combination of materials. Dissolvable supports as described herein are supports that may be in any suitable format to provide a protected environment for the culturing of cells where the cell-to-cell interactions and formation of extracellular matrix (ECM) in a 3D fashion are aided. The dissolvable supports may be completely digested which allows for harvesting of cells without damaging the cells when using protease treatment and/or mechanical harvesting techniques. Supports prepared according to embodiments of the disclosure allow for highly efficient release of the cells that are captured or cultured in the support by means of dissolution of the support using non-proteolytic enzymes, chelating agents, or both.

In some embodiments, dissolvable supports as described herein are digested when exposed to an appropriate enzyme that digests or breaks down the material. Methods for harvesting cells as described herein may include digesting the dissolvable supports by exposing the dissolvable supports to an enzyme. Non-proteolytic enzymes suitable for digesting the supports, harvesting cells, or both, include pectinolytic enzymes or pectinases, which are a heterogeneous group of related enzymes that hydrolyze the pectic substances. Pectinases (polygalacturonase) are enzymes that break down complex pectin molecules to shorter molecules of galacturonic acid. Commercially available sources of pectinases are generally multi-enzymatic, such as Pectinex™ ULTRA SP-L (commercially available from Novozyme North American, Inc., Franklinton, NC), a pectolytic enzyme preparation produced from a selected strain of Aspergillus aculeatus. Pectinex™ ULTRA SP-L contains mainly polygalacturonase, (EC 3.2.1.15), pectintranseliminase (EC 4.2.2.2), and pectinesterase (EC: 3.1.1.11). The EC designation is the Enzyme Commission classification scheme for enzymes based on the chemical reactions the enzymes catalyze.

Exposing the dissolvable supports to an enzyme may include exposing the supports to enzyme concentrations of between about 1 and about 200 U. For example, the method may include exposing the supports to enzyme concentrations of between about 2 U and about 150 U, or between about 5 U and about 100 U, or even between about 10 U and about 75 U, and all values therebetween.

Methods for harvesting cells as described herein may further include exposing the material to a chelating agent. In some embodiments, dissolvable supports as described herein are digested when exposed to an appropriate chelating agent that digests or breaks down the material. According to embodiments of the present disclosure, digestion of the dissolvable supports includes exposing the supports to a divalent cation chelating agent. Exemplary chelating agents include, but are not limited to, ethylenediaminetetraacetic acid (EDTA), cyclohexanediaminetetraacetic (CDTA), ethylene glycol tetraacetic acid (ETGA), citric acid and tartaric acid.

Exposing the dissolvable supports to a chelating agent may include exposing the supports to chelating agent concentrations of between about 1 mM and about 200 mM. For example, the method may include exposing the supports to chelating agent concentrations of between about 10 mM and about 150 mM, or between about 20 mM and about 100 mM, or even between about 25 mM and about 50 mM, and all values therebetween.

The time to complete digestion of dissolvable supports as described herein may be less than about 1 hour. For example, the time to complete digestion of supports may be less than about 45 minutes, or less than about 30 minutes, or less than about 15 minutes, or between about 1 minute and about 25 minutes, or between about 3 minutes and about 20 minutes, or even between about 5 minutes and about 15 minutes.

### EXAMPLES

Embodiments of the present disclosure are further described below with respect to certain exemplary and specific embodiments thereof, which are illustrative only and not intended to be limiting.

### Example 1

Example 1 compares a traditional ligand binding method with a binding method according to embodiments of the disclosure, as shown below in Scheme I and Scheme II. Scheme I is an example of a conventional or traditional way to bind a ligand to a material such as alginic acid or polygalacturonic acid. Scheme II is an example of a method of binding ligands according to embodiments of the disclosure.

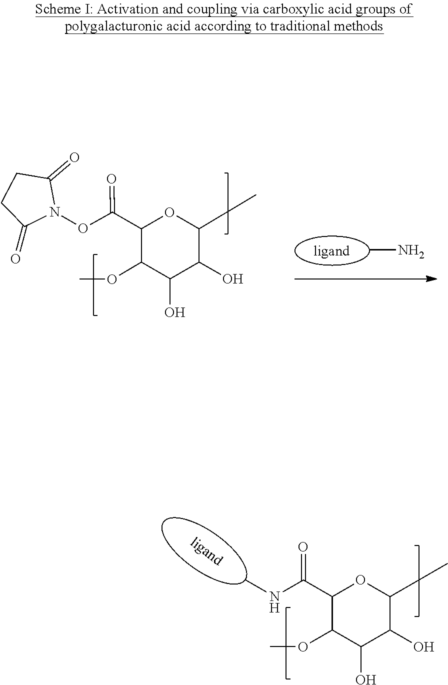

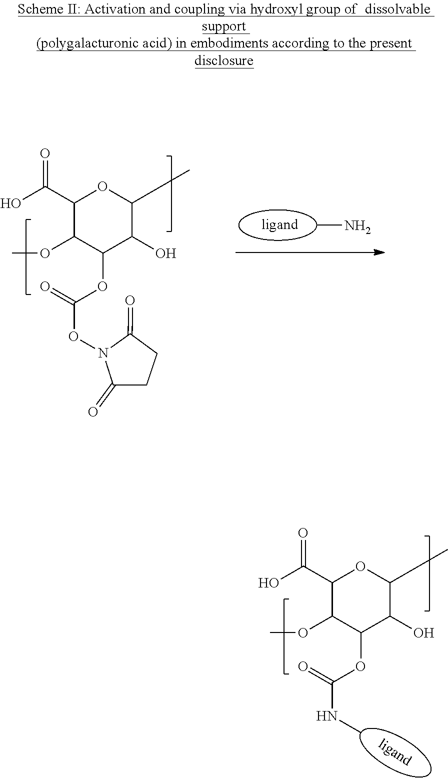

As shown in the traditional method depicted in Scheme I, the carboxylic acid groups are activated, and then the ligands are bound to such activated carboxylic acids groups (NHS ester). When the derivatized carboxylic acids are converted into amide linkers, they are no longer able to contribute to ionic crosslinking with calcium ions, such as free-carboxylic acids can ionically crosslink. Therefore, the crosslinking density is lowered, which results in degradation of the mechanical properties of the dissolvable support.

In contrast, as shown in the method according to embodiments of the disclosure depicted in Scheme II, the ligand is bound to hydroxyl groups while the carboxylic acid groups are untouched. Therefore, the carboxylic acid groups remain available for ionic crosslinking by multivalent cations, e.g. calcium. Consequently, the mechanical properties of the dissolvable support are almost unchanged since the crosslinking density is not lowered regardless the amount of bound ligands.

### Example 2

Example 2 illustrates the activation of macroporous PGA-based dissolvable foam. A macroporous support was prepared as follows.

A 2 wt % PGA solution, about 162 g, was prepared by dissolving the appropriate amount of Polygalacturonic acid sodium salt (PGA), available from Sigma Aldrich, in demineralized water using an oil bath (104° C. setting temp). The resulting solution was cooled to room temperature. To this solution, 0.97 g Pluronic 123 was added, which was dissolved at a low temperature under mixing. The resulting solution was then placed in a mixer bowl (e.g., KitchenAid mixer bowl). Then, 17.5 g sucrose and 7.5 g glycerol were added to the bowl, and the mixture was gently mixed until total dissolution of the sugar. A solution prepared from 0.97 g 150 kDa Dextran and 0.125 g Tween™ 20 in 10 ml water was added to the bowl and gently mixed to achieve a homogenous mixture. Then, a dispersion made of 0.53 g CaCO3 (Sigma), 0.248 g Sodium dodecyl sulfate, and 14.5 ml ultra-pure (UP) water was added to the bowl. Foams were prepared by whipping the suspension using a wire loop whip at speed 2 for 5 minutes to incorporate air. After that, a freshly made Glucono delta-lactone (GDL) solution prepared by mixing 3.77 g GDL and 12.6 g DMSO was quickly added into the bowl, and whipping was continued for about 60 seconds. The foam was left to rest uncovered for 3 hours in the bowl on the bench at 23ºC to allow completion of the crosslinking. Then, the crosslinked foam was punched and sliced in the form of discs (slices).

Afterward, the pieces of foam were frozen at −80ºC for 16 hours prior to freeze-drying at −86° C. under 0.11 mbar for 72 hrs. The resulting foam exhibits a dry foam density (DFD) of about 0.04-0.045 g/cc.

The foam slices were then activated by reaction with DSC as follow. Briefly, 10 slices, 34 mg each, were added to a 50 ml plastic tube and were rinsed two times with UP water and three times with anhydrous DMSO to remove unbound materials. About 90% of the material being additives are removed from the foam. Then, the supernatant was discarded and about 40 ml of a solution prepared by mixing 40 ml anhydrous DMSO, 394 mg N,N′-Disuccinimidyl carbonate (DSC) and 167 mg 4-Dimethylaminopyridine (DMAP) was added to the slices. The tube was placed on a shaker and agitated for 5.5 hours at room temperature (RT). The slices were rinsed two times with 4 wt % CaCl₂) pH 8.5 to remove unreacted reagents. The foam can be freeze-dried at this step and stored at 4° C. in the dark with desiccant for months.

### Example 3

Example 3 illustrates the coupling of VN peptide on a dissolvable support from Example 2.

Four (4) ml of a 5 mg/ml Vitronectin-NH2 peptide (from American Peptide Company, Inc) solution in 4% CaCl2 adjusted at pH 9 was added per foam slice. The slices were incubated for 1 night at 60° C. in an oven. The slices were thoroughly rinsed with UP water, and the amount of immobilized VN peptide was quantified using bicinchoninic acid assay (BCA assay). The BCA analysis showed that about 0.5 μg peptide was immobilized per mg of dry foam. The foam resisted extensive rinsing without any signs of deterioration.

### Example 4

Example 4 illustrates the immobilization of Protein A on an activated dissolvable support prepared as described in Example 2, except that 130 mg DSC and 62 mg DMAP were used and the activation was performed for 18 hours.

Four (4) ml of a 0.25 mg/ml Protein A solution in 4 wt % CaCl2 pH 9 was added per foam slice prepared according to Example 2. The slices were incubated for 48 hours at RT and thoroughly rinsed one time with 1 wt % Tergitol™ NP40 in Dulbecco's phosphate-buffered saline (DPBS) solution and then rinsed three times with DPBS. The amount of immobilized protein A was quantified using bicinchoninic acid assay (BCA assay). The BCA analysis showed that about 0.9 μg protein A was immobilized per mg of dry foam. The foam resisted extensive washing.

To show that the immobilized protein A was functional, OKT3 murine monoclonal antibody was bound to the PA-functionalized foam. In a typical experiment, a solution containing 160 μg OKT3 antibody in about 8 ml DPBS was prepared. To each PA functionalized foam slice, 4 ml of the OKT3 antibody solution was added. The slice was left to rest for 1 hour at RT and then rinsed with 2 ml 1% Tergitol NP40 in DPBS followed by 3 rinses with 2 ml DPBS. Elisa assay showed that about 0.75 μg OKT3 antibody was immobilized per slice.

### Example 5

Example 5 illustrates the culture of HEK293T cells in the porous structure of the vitronectin peptide (VN)-functionalized foam activated according to the embodiment described in Example 3. Such VN peptide-functionalized foams are referred to as “VN” within Example 5.

As a negative control, slices were activated according to the embodiment described in Example 2 but were blocked with ethanolamine instead of being functionalized with vitronectin. Such blocked foams are referred to as “EA” within Example 5.

Both VN and EA functionalized foam slices as prepared as described above according to Example 3 were washed as follows. The slices were placed in the wells of a polystyrene 6 well plate. Four (4 ml) 70% aqueous ethanol was added to each well for sanitization for 10 minutes. Then, the aqueous ethanol solution was removed by aspiration and the foam was rinsed 3 times with UP water.

After removal of the excess of water, the wet foam slices were transferred in the wells of a Costar® 6-well Clear Flat Bottom Ultra-Low Attachment Multiple Well Plate, Product Number 3471 (Corning Incorporated, Corning, NY) containing 4 ml complete Iscove's Modified Dulbecco's Medium (IMDM). The complete IMDM medium was prepared by mixing 450 ml IMDM, 50 ml FBS, 5 ml Penicillin/streptomycin, and 5 ml Glutamax™ (commercially available from Thermo Fisher Scientific).

Once the foam slices absorbed the medium, the excess of medium was removed by aspiration. Then, 150 μl of medium containing 300 K HEK293 T cells were added to each slice. The plate was left undisturbed for 3 hours in an incubator at 37° C./5% CO2 to allow the cells to adhere. Then, 4 ml of complete medium was added, and the cells were left to grow for 5 days.

Attachment of the cells was evaluated 1 day after seeding by visual inspection using a fluorescence microscope after calcein staining. Cell counting was performed by digesting the foam scaffold 5 days after seeding by adding a solution consisting of 0.5 ml trypsin, 2 ml pectinase 50 U and 5 mM EDTA on each slice. Dissolution took about 5 min.

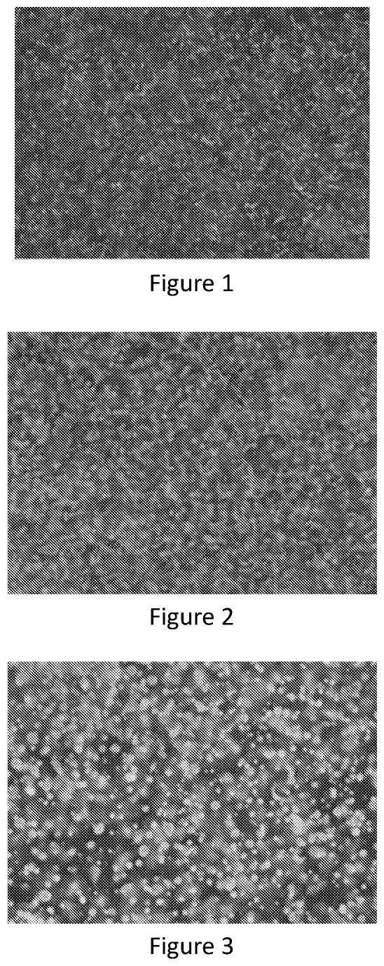

FIG. 1 shows spreading and attachment of the HEK293 T cells seeded in the VN-functionalized scaffold according to Example 5 (VN). FIG. 2 shows the cells covering the whole surface of the scaffold five days after seeding. Counting showed that the cells were expanded by 6.5-fold.

FIG. 3 shows a fluorescent image of the negative control. In contrast to the attachment shown in FIG. 1 and FIG. 2, the cells are not able to adhere to the negative control foam that was blocked with ethanolamine (EA). Instead, the cells aggregated in clusters in FIG. 3 because they were unable to adhere to the control foam blocked with EA.

### Example 6

Example 6 illustrates the capture of Jurkat cell expressing CD3 antigens by a foam scaffold functionalized with an OKT3 antibody.

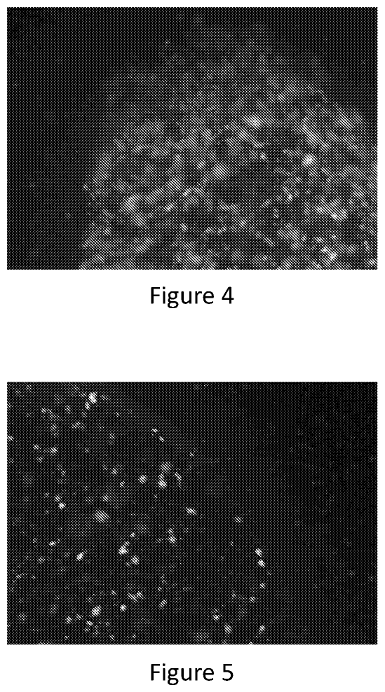

OKT3-bound foam slices prepared according to Example 4 were transferred in wells of a Costar® 6-well Clear Flat Bottom Ultra-Low Attachment Multiple Well Plate, Product Number 3471 (Corning Incorporated, Corning, NY) along with 4 ml of medium prepared by mixing 450 ml Roswell Park Memorial Institute medium (ATCC-modified RPMI), 50 ml FBS, 5 ml Penicillin/streptomycin, and 5 ml Glutamax™ (commercially available from Thermo Fisher Scientific). The excess of medium was removed by aspiration as much as possible. Then, 150 μl of Jurkat cell suspension, 10,000 K (10 million)/ml, was added on each piece of scaffold. The scaffolds were incubated for 30 min at 37° C. to allow for cell capture. The foam was then washed 3 times with D-PBS buffer after staining the cells with calcein. The stained cells were imaged. As shown by the stained cells in FIG. 4, numerous cells were captured by the OKT3-bound scaffold.

Protein A-functionalized scaffolds, free of OKT3, were used as the negative control (referred to within Example 6 as “PA”). The cells were stained with calcein and imaged. The image of FIG. 5 shows that, in contrast to FIG. 4, only a small amount of cells was nonspecifically trapped in the negative control (PA-functionalized support exempt of OKT3).

### Comparative Example 1

Comparative Example 1 illustrates that the activation of the carboxylic acid groups can have a negative effect on the crosslinking and dissolvable support stability.

A foam slice was prepared using the method described in Example 2, as follows. A 2 wt % PGA solution, about 162 g, was prepared by dissolving the appropriate amount of Polygalacturonic acid sodium salt (PGA), available from Sigma Aldrich, in demineralized water using an oil bath (104° C. setting temp). The resulting solution was cooled to room temperature. To this solution, 0.97 g Pluronic 123 was added, which was dissolved at a low temperature under mixing. The resulting solution was then placed in a mixer bowl (e.g., KitchenAid mixer bowl). Then, 17.5 g sucrose and 7.5 g glycerol were added to the bowl, and the mixture was gently mixed until total dissolution of the sugar. A solution prepared from 0.97 g 150 kDa Dextran and 0.125 g Tween™ 20 in 10 ml water was added to the bowl and gently mixed to achieve a homogenous mixture. Then, a dispersion made of 0.53 g CaCO3 (Sigma), 0.248 g Sodium dodecyl sulfate, and 14.5 ml ultra-pure (UP) water was added to the bowl. Foams were prepared by whipping the suspension using a wire loop whip at speed 2 for 5 minutes to incorporate air. After that, a freshly made Glucono delta-lactone (GDL) solution prepared by mixing 3.77 g GDL and 12.6 g DMSO was quickly added into the bowl, and whipping was continued for about 60 seconds. The foam was left to rest uncovered for 3 hours in the bowl on the bench at 23° C. to allow completion of the crosslinking. Then, the crosslinked foam was punched and sliced in the form of discs (slices). Afterward, the pieces of foam were frozen at −80° ° C. for 16 hours prior to freeze-drying at −86° C. under 0.11 mbar for 72 hrs. The resulting foam exhibits a dry foam density (DFD) of about 0.04-0.045 g/cc.

In Example 2, the prepared foam slice was then activated by DSC. In contrast, the prepared foam slice in Comparative Example 1 was instead activated by reaction with 1-ethyl-3-(3-dimethylaminopropyl) carbodiimide (EDC)/N-hydroxysuccinimide (NHS).

The EDC/NHS activation was performed as follows. The foam slices were rinsed 4 times with 4 ml UP water to remove unbound materials. Then, the scaffolds were activated by adding 4 ml 200 mM EDC and 50 mM NHS. Activation was performed for 30 min at RT and then the scaffolds were rinsed one time with Dulbecco's phosphate-buffered saline (DPBS). The excess of DPBS was carefully removed by aspiration.

Next, protein A was immobilized using conditions described in Example 4.

By changing the activation reaction and activating agent, the foam slice prepared in Comparative Example 1 has activated carboxyl groups instead of having activated hydroxyl groups as described in Example 2. In contrast to Example 2, the resulting foam slice in Comparative Example 1 disintegrated during the rinsing process, which proves that the consumption of carboxyl groups to yield amide bonds has a deleterious effect on the foam stability due to the reduction of the density of ionic crosslinking sites.

It will be appreciated that the various disclosed embodiments may involve particular features, elements, or steps that are described in connection with that particular embodiment. It will also be appreciated that a particular feature, element, or step, although described in relation to one particular embodiment, may be interchanged or combined with alternate embodiments in various non-illustrated combinations or permutations.

It is also to be understood that, as used herein the terms “the,” “a,” or “an,” mean “at least one,” and should not be limited to “only one” unless explicitly indicated to the contrary. Thus, for example, reference to “an opening” includes examples having two or more such “openings” unless the context clearly indicates otherwise.

All scientific and technical terms used herein have meanings commonly used in the art unless otherwise specified. The definitions provided herein are to facilitate understanding of certain terms used frequently herein and are not meant to limit the scope of the present disclosure.

As used herein, “have,” “having,” “include,” “including,” “comprise,” “comprising,” or the like are used in their open-ended sense, and generally mean “including, but not limited to.”

Ranges may be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All numerical values expressed herein are to be interpreted as including “about,” whether or not so stated, unless expressly indicated otherwise. It is further understood, however, that each numerical value recited is precisely contemplated as well, regardless of whether it is expressed as “about” that value. Thus, “a dimension less than 10 mm” and “a dimension less than about 10 mm” both include embodiments of “a dimension less than about 10 mm” as well as “a dimension less than 10 mm.”

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising A+B+C include embodiments where a method consists of A+B+C, and embodiments where a method consists essentially of A+B+C.

### Illustrative Implementations

The following is a description of various aspects of implementations of the disclosed subject matter. Each aspect may include one or more of the various features, characteristics, or advantages of the disclosed subject matter. The implementations are intended to illustrate a few aspects of the disclosed subject matter and should not be considered a comprehensive or exhaustive description of all possible implementations.

Aspect 1 pertains to an activated dissolvable support including an ionotropically crosslinked compound comprising a polymer material having at least one repeating unit including: an ionically crosslinked carboxylic acid group, and an activated hydroxyl group, wherein the hydroxyl group is activated by N,N′-disuccinimidyl carbonate (DSC) or N-hydroxysuccinimidyl chloroformate in a solvent to form succinimidyl carbonate groups for ligand binding.

Aspect 2 pertains to the dissolvable support of Aspect 1, wherein the carboxylic acid group is ionically crosslinked with a multivalent cation.

Aspect 3 pertains to the dissolvable support of Aspect 1, wherein the at least one repeating unit includes:

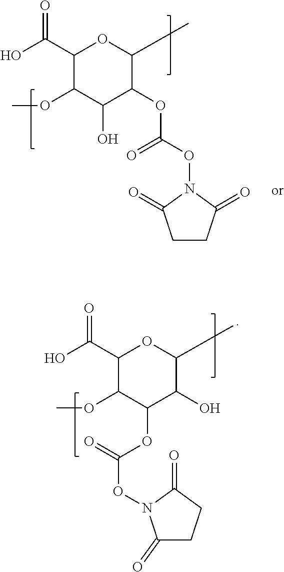

Aspect 4 pertains to the dissolvable support of Aspect 1, wherein the solvent is an aprotic solvent.

Aspect 5 pertains to the dissolvable support of Aspect 4, wherein the aprotic solvent is an anhydrous solvent.

Aspect 6 pertains to the dissolvable support of Aspect 1, wherein the polymer material comprises a polygalacturonic acid (PGA) compound.

Aspect 7 pertains to the dissolvable support of Aspect 1, wherein the PGA compound comprises at least one of pectic acid, partially esterified pectic acid, partially amidated pectic acid, or salts thereof.

Aspect 8 pertains to the dissolvable support of Aspect 1, wherein the partially esterified pectic acid comprises a degree of esterification from 1 mol % to 40 mol %.

Aspect 9 pertains to the dissolvable support of Aspect 1, wherein the partially amidated pectic acid comprises a degree of amidation from 1 mol % to 40 mol %.

Aspect 10 pertains to the dissolvable support of Aspect 1, wherein the dissolvable support comprises a structure comprising beads, fibers, fabric, foam, or a coating.

Aspect 11 pertains to the dissolvable support of Aspect 10, wherein the dissolvable support comprises porous beads.

Aspect 12 pertains to the dissolvable support of Aspect 10, wherein the dissolvable support comprises a macroporous foam.

Aspect 13 pertains to the dissolvable support of Aspect 10, wherein the dissolvable support comprises a coating for cell culture surfaces of cell culture vessels.

Aspect 14 pertains to the dissolvable support of Aspect 1, wherein the dissolvable support is dissolved by digestion from an enzyme, chelating agent, or a combination thereof.

Aspect 15 pertains to the dissolvable support of Aspect 1, wherein the enzyme comprises a non-proteolytic enzyme.

Aspect 16 pertains to the dissolvable support of Aspect 1, wherein the non-proteolytic enzyme is selected from the group consisting of pectinolytic enzymes and pectinases.

Aspect 17 pertains to the dissolvable support of Aspect 1, wherein digestion of the dissolvable support is complete in less than about 1 hour.

Aspect 18 pertains to the dissolvable support of Aspect 1, wherein digestion of the dissolvable support is complete in less than about 15 minutes.

Aspect 19 pertains to the dissolvable support of Aspect 1, wherein the ligand comprises a protein, peptide, peptoid, sugar, or drug.

Aspect 20 pertains to a method of forming an activated dissolvable support including: forming an ionotropically crosslinked compound. The forming the ionotropically crosslinked compound includes forming a dissolvable support by adding a polymer solution with a polygalacturonic acid (PGA) compound to a solution with at least one multivalent cation, the PGA compound selected from at least one of: pectic acid, partially esterified pectic acid, partially amidated pectic acid and salts thereof; and activating the hydroxyl groups to form succinimidyl carbonate groups in the PGA compound of the dissolvable support by adding an activation solution.

Aspect 21 pertains to the method of Aspect 20, wherein the method further comprises rinsing the dissolvable support before activation to remove unbound hydroxyl-containing compounds.

Aspect 22 pertains to the method of Aspect 20, wherein the method further comprises coupling ligands to the activated hydroxyl groups to create a ligand-dissolvable support conjugate.

Aspect 23 pertains to the method of Aspect 20, wherein the ligand-dissolvable support conjugate comprises at least one unit comprising:

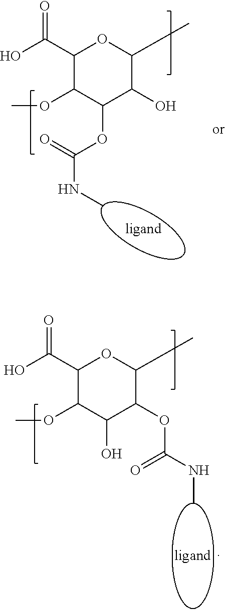

Aspect 24 pertains to the method of Aspect 20, wherein the ligand comprises proteins, peptides, peptoids, sugars, and drugs.

Aspect 25 pertains to the method of Aspect 20, wherein the method further comprises rinsing the activated dissolvable support after activation with a solution that contains at least one multivalent cation.

Aspect 26 pertains to the method of Aspect 20, wherein the ionotropically crosslinked compound can be formed into a structure comprising beads, fibers, fabric, or a foam.

Aspect 27 pertains to the method of Aspect 20, wherein the ionotropically crosslinked compound can be applied to a cell culture surface as a coating.

Aspect 28 pertains to the method of Aspect 20, wherein the activation solution comprises an activation agent and a solvent.

Aspect 29 pertains to the method of Aspect 20, wherein the activation agent comprises N,N′-disuccinimidyl carbonate (DSC) or N-hydroxysuccinimidyl chloroformate.

Aspect 30 pertains to the method of Aspect 20, wherein the solvent comprises an aprotic solvent.

Aspect 31 pertains to the method of Aspect 20, wherein the polygalacturonic acid compound comprises at least one of pectic acid, partially esterified or amidated pectic acid having a degree of esterification or amidation from 1 to 40 mol %, or salts thereof.

Aspect 32 pertains to the method of Aspect 20, wherein the polygalacturonic acid compound contains less than 20 mol % methoxyl groups.

Aspect 33 pertains to a method for culturing cells on a dissolvable support including: seeding cells on a dissolvable support; and contacting the dissolvable support with cell culture medium.

Aspect 34 pertains to the method of Aspect 33, wherein seeding cells on a dissolvable support comprises adhering cells to the surface of the dissolvable support.

Aspect 35 pertains to the method of Aspect 33, wherein cells aggregate in pores of the dissolvable support to form spheroids.

Aspect 36 pertains to the method of Aspect 33, wherein contacting the dissolvable support with cell culture medium comprises submerging the dissolvable support in cell culture medium.

Aspect 37 pertains to the method of Aspect 33, wherein contacting the dissolvable support with cell culture medium comprises continuously passing cell culture medium over the dissolvable support.

Aspect 38 pertains to the method of Aspect 37, wherein continuously passing cell culture medium over the dissolvable support comprises removing at least some of the cell culture medium from contact with the dissolvable support and contacting the dissolvable support with fresh cell culture medium such that the volume of cell culture medium in contact with the dissolvable support remains substantially constant.

Aspect 39 pertains to a method of harvesting cells from a dissolvable support, the method including: digesting the dissolvable support by exposing the dissolvable foam scaffold to an enzyme, chelating agent, or a combination thereof; and harvesting cells exposed when the dissolvable support is digested.

Aspect 40 pertains to the method of Aspect 39, wherein the dissolvable support comprises an ionotropically crosslinked polygalacturonic acid compound selected from at least one of: pectic acid; partially esterified pectic acid, partially amidated pectic acid and salts thereof, and wherein the enzyme comprises a non-proteolytic enzyme.

Aspect 41 pertains to the method of Aspect 40, wherein the non-proteolytic enzyme is selected from the group consisting of pectinolytic enzymes and pectinases.

Aspect 42 pertains to the method of Aspect 39, wherein digesting the dissolvable support comprises exposing the dissolvable support to between about 1 U and about 200 U of the enzyme.

Aspect 43 pertains to the method of Aspect 39, wherein digesting the dissolvable support comprises exposing the dissolvable support to between about 1 mM and about 200 mM of the chelating agent.

Although multiple embodiments of the present disclosure have been described in the Detailed Description, it should be understood that the disclosure is not limited to the disclosed embodiments, but is capable of numerous rearrangements, modifications and substitutions without departing from the disclosure as set forth and defined by the following claims.

1. An activated dissolvable support comprising:
an ionotropically crosslinked compound comprising a polymer material having at least one repeating unit comprising:
an ionically crosslinked carboxylic acid group, and
an activated hydroxyl group, wherein the hydroxyl group is activated by N,N′-disuccinimidyl carbonate (DSC) or N-hydroxysuccinimidyl chloroformate in a solvent to form succinimidyl carbonate groups for ligand binding.

2. The dissolvable support of claim 1, wherein the carboxylic acid group is ionically crosslinked with a multivalent cation.

3. The dissolvable support of claim 1, wherein the at least one repeating unit comprises:

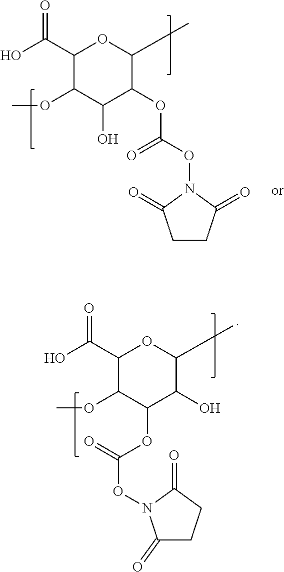

4. The dissolvable support of claim 1, wherein the solvent is an aprotic solvent.

5. The dissolvable support of claim 4, wherein the aprotic solvent is an anhydrous solvent.

6. The dissolvable support of claim 1, wherein the polymer material comprises a polygalacturonic acid (PGA) compound.

7. The dissolvable support of claim 6, wherein the PGA compound comprises at least one of pectic acid, partially esterified pectic acid, partially amidated pectic acid, or salts thereof.

8. The dissolvable support of claim 7, wherein the partially esterified pectic acid comprises a degree of esterification from 1 mol % to 40 mol %.

9. The dissolvable support of claim 7, wherein the partially amidated pectic acid comprises a degree of amidation from 1 mol % to 40 mol %.

10. The dissolvable support of claim 1, wherein the dissolvable support comprises a structure comprising beads, fibers, fabric, foam, or a coating.

11. (canceled)

12. (canceled)

13. The dissolvable support of claim 10, wherein the dissolvable support comprises a coating for cell culture surfaces of cell culture vessels.

14-18. (canceled)

19. The dissolvable support of claim 1, wherein the ligand comprises a protein, peptide, peptoid, sugar, or drug.

20. A method of forming an activated dissolvable support comprising:
forming an ionotropically crosslinked compound comprising:
forming a dissolvable support by adding a polymer solution comprising a polygalacturonic acid (PGA) compound to a solution comprising at least one multivalent cation, the PGA compound selected from at least one of: pectic acid, partially esterified pectic acid, partially amidated pectic acid and salts thereof; and
activating the hydroxyl groups to form succinimidyl carbonate groups in the PGA compound of the dissolvable support by adding an activation solution.

21. The method of claim 20, wherein the method further comprises rinsing the dissolvable support before activation to remove unbound hydroxyl-containing compounds.

22. The method of claim 20, wherein the method further comprises coupling ligands to the activated hydroxyl groups to create a ligand-dissolvable support conjugate.

23. The method of claim 22, wherein the ligand-dissolvable support conjugate comprises at least one unit comprising:

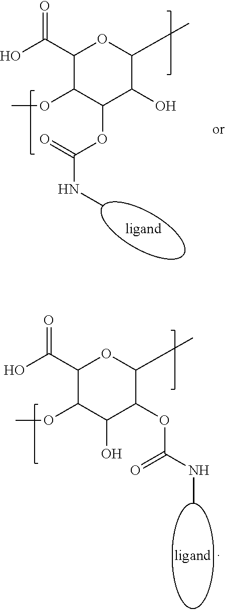

24. The method of claim 22, wherein the ligand comprises proteins, peptides, peptoids, sugars, and drugs.

25. The method of claim 20, wherein the method further comprises rinsing the activated dissolvable support after activation with a solution that contains at least one multivalent cation.

26. The method of claim 20, wherein the ionotropically crosslinked compound can be formed into a structure comprising beads, fibers, fabric, or a foam.

27. (canceled)

28. The method of claim 20, wherein the activation solution comprises an activation agent and a solvent.

29. The method of claim 28, wherein the activation agent comprises N,N′-disuccinimidyl carbonate (DSC) or N-hydroxysuccinimidyl chloroformate.

30-43. (canceled)
